(19) United States
(12) Patent Application Publication
(10) Pub. No.: US 2025/0302821 A1
(43) Pub. Date: Oct. 2, 2025

(54) SYNERGISTIC ANTIFUNGAL COMPOSITION AND METHOD

(71) Applicant: ST. JUDE CHILDREN'S RESEARCH HOSPITAL, INC., Memphis, TN (US)

(72) Inventor: Jeffrey M. RYBAK, Memphis, TN (US)

(21) Appl. No.: 18/864,218

(22) PCT Filed: May 11, 2023
(86) PCT No.: PCT/US2023/021801

§ 371 Date: Nov. 8, 2024

Related U.S. Application Data
(60) Provisional application No. 63/341,066, filed on May 12, 2022.

(51) Int. Cl. A61K 31/496; A01N 43/653; A01N 43/90; A01P 3/00; A61K 31/438

(52) U.S. Cl. CPC A61K 31/496; A01N 43/653; A01N 43/90; A01P 3/00; A61K 31/438

(57) ABSTRACT

This invention provides a method for inhibiting the growth of a pathogenic mold by contacting the pathogenic mold with a synergistically effective amount of an NK1 antagonisfand an antifungal agent. This invention also provides a method for inhibiting the growth of a mammalian fungal pathogen by contacting the mammalian fungal pathogen with a synergistically effective amount of posaconazole and rolapitant. In some aspects, the mammalian fungal pathogen is a species of Candida (e.g., C. glabrata, C. auris, C. albicans, C. dubliniensis, or C. parapsilosis). Aspergillus, Cryptococcus, Mucor, orRhizopus. In addition, A surface disinfectant comprising a synergistic amount of an NK1 antagonist and an antifungal agent in admixture with a carrier or excipient.

### INTRODUCTION

This patent application claims the benefit of priority from U.S. Provisional Ser. No. 63/341,066, filed May 12, 2022, the content of which is incorporated herein by reference in its entirety.

### BACKGROUND

The incidence of invasive fungal infections has been dramatically increasing over the years. In particular, Candida species (spp.) are the fourth most common cause of nosocomial bloodstream infections in the United States. Other fungal pathogens such as Aspergillus spp., zygomycetes, Fusarium spp., and Scedosporium spp. have become more common at causing invasive infections. There are multiple reasons for this increased incidence, including the use of broad-spectrum antibiotics, central venous catheters, and prosthetic devices. Additionally, patients with burns and neutropenia or those who are the recipients of parenteral nutrition, renal replacement therapy, immunosuppressive therapy, and antineoplastic agents can also be predisposed to fungal infections. Invasive fungal infections can have a significant impact on patient morbidity and mortality.

Azole antifungals have been used in clinical practice to treat various fungal infections. They are categorized into three distinct classes: the imidazoles, the tetrazoles, and the triazoles. The imidazoles include several agents, most notably clotrimazole, ketoconazole, and miconazole. The tetrazoles include oteseconazole and VT-1598. The triazoles include fluconazole, itraconazole, terconazole, voriconazole, isavuconazole, and posaconazole. As a class, they exert their effect by impairing the synthesis of ergosterol, a vital component in the fungal cellular membrane. This effect occurs through the inhibition of CYP450, which converts lanosterol to ergosterol, resulting in increased cellular permeability and leakage of cellular contents as well as inhibition of fungal growth. However, strains resistant to these antifungal agents have been identified.

The use of aprepitant (a neurokinin receptor subtype-1 antagonist) has been shown to restore fluconazole-, voriconazole-, and itraconazole-susceptibility in several resistant Candida auris strains and synergistically enhance the activity of these triazole antifungals against C. auris (Eldesouky et al. (2020) Virulence 11 (1):1466-1481). However, given the increase in the occurrence of drug-resistant fungal strains, especially in the context of common species of Aspergillus spp., as well as rare and hard-to-treat molds including Fusarium spp., Scedosporium spp., and molds from the Mucorales order, there is still a need in the art for new antifungal therapies against these fungal pathogens.

### SUMMARY OF THE INVENTION

This invention provides a method for inhibiting the growth of a pathogenic mold by contacting the pathogenic mold with a synergistically effective amount of an NK1 antagonist and an antifungal agent thereby inhibiting the growth of the pathogenic mold. In some aspects, the antifungal agent is a sterol biosynthesis inhibitor, e.g., a triazole.

This invention also provides a method for inhibiting the growth of a mammalian fungal pathogen by contacting the mammalian fungal pathogen with a synergistically effective amount of posaconazole and rolapitant. In some aspects, the mammalian fungal pathogen is a species of Candida (e.g., C. glabrata, C. auris, C. albicans, C. dubliniensis, or C. parapsilosis), Aspergillus, Cryptococcus, Mucor, or Rhizopus.

A surface disinfectant comprising, consisting of, or consisting essentially of a synergistic amount of an NK1 antagonist and an antifungal agent in admixture with a carrier or excipient is also provided.

### DETAILED DESCRIPTION OF THE INVENTION

This invention provides a synergistic combination of antifungal agents, notably most sterol biosynthesis inhibitors, and an antagonist of the neurokinin receptor subtype-1 (“NK1 antagonist”) class of antiemetic agents, for inhibiting the growth of fungi. The synergistic effect is observed against pathogenic yeast, in particular antifungal-resistant clinical isolates of Candida, as well as pathogenic molds. In particular, the combination of agents enhances the ability of triazole antifungals to kill fungi rather than just inhibit growth. Notably, the combination of posaconazole and rolapitant were particularly effective at killing three different representative clinical isolates of Aspergillus fumigatus, with an 8-fold potency over the antifungal alone. In addition, the combination of posaconazole and rolapitant also exhibited enhanced activity against triazole-resistant isolates. Furthermore, the combination of rolapitant and posaconazole demonstrated synergy against Candida glabrata as well as C. auris, C. albicans, C. dubliniensis, and C. parapsilosis. Moreover, it has been found that select NK1-antagonists, such as netupitant and rolapitant, independently exert fungicidal activity at higher concentrations. Therefore, this invention provides methods for inhibiting the growth of pathogenic fungi, including yeast and molds, with a synergistically effective amount of an NK1 antagonist and an antifungal agent. The methods of this invention find use in restoring the use of the triazole antifungals and enhancing the treatment of patients with difficult to treat fungal infections such as those caused by Candida spp., Aspergillus spp., Cryptococcus spp., Mucor spp., Rhizopus spp., and the like.

In general, the methods of this invention provide for contacting a pathogenic fungus with a synergistically effective amount of an NK1 antagonist and an antifungal agent thereby inhibiting the growth of the pathogenic fungus. As used herein, “contacting” refers to any suitable means for delivering, or exposing, an agent to at least one fungal cell. Exemplary delivery methods include, but are not limited to, direct delivery to cell culture medium, perfusion, injection, or other delivery method well known to one skilled in the art. In some aspects, contacting includes physical human activity, e.g., an injection; an act of dispensing, mixing, and/or decanting; and/or manipulation of a delivery device or machine.

The term “effective amount” means an amount of a composition sufficient to decrease the growth of a fungal pathogen and/or provide at least some amelioration of the symptoms associated with a fungal infection. A “synergistically effective amount” of a composition refers to the amount of one component necessary to elicit a synergistic effect in another component present in the composition. Thus, the term “synergy,” and derivations thereof, refers to a substance that enhances the activity of an active ingredient, in the present case an antifungal agent. In certain aspects of this invention, the composition of the invention includes a synergistically effective amount of the NK1 antagonist and a synergistically effective amount of the antifungal agent, wherein at least one of: i) the synergistically effective amount of the NK1 antagonist is lower than an amount of NK1 antagonist required to decrease the growth of a fungal pathogen and/or provide at least some amelioration of the symptoms associated with a fungal infection in the absence of the antifungal agent; ii) the synergistically effective amount of the antifungal agent is lower than an amount of the antifungal agent required to decrease the growth of a fungal pathogen and/or provide at least some amelioration of the symptoms associated with a fungal infection in the absence of the NK1 antagonist; and (iii) combinations thereof.

The efficacy or growth inhibitory effect of the synergistic composition herein can be assessed in any suitable in vitro or animal model assays. By way of non-limiting example, the effects of a dose of a synergistic composition including at least one antifungal agent and at least one NK1-anatgonist can be assessed using a microdilution-broth based antifungal susceptibility testing (AFST) provided by the Clinical and Laboratory Standards Institute (CLSI; formerly the National Committee for Clinical Laboratory Standards) and The European Committee on Antimicrobial Susceptibility Testing (EUCAST). AFST methods may be used for testing the activity of antifungal agents against yeasts (the CLSI M27, M44, M60 and the EUCAST E. Def 7.3 documents) and filamentous fungi (molds; the CLSI M38, M44, M51, M61 and EUCAST E. Def 9.3 documents). These reference AFST methods, or their commercial counterparts such as Sensititre YeastOne (SYO, Thermo Fisher Scientific, MA) rely on measuring growth of a defined fungal inoculum in a specific growth broth in the presence of different concentrations of the antifungal drug and allow the determination of the MIC (the minimum inhibitory concentration) of the drug resulting in complete or prominent growth inhibition.

In certain aspects, the synergistic composition of the invention “inhibits,” “decreases”, or “reduces” the growth of a pathogenic fungus by a statistically significant amount. In some aspects, inhibit, decrease, or reduce means a decrease by at least 10% as compared to a reference level (e.g., the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” also encompasses complete inhibition or reduction as compared to a reference level.

Ideally, an antifungal agent of use in the compositions and methods described herein is “fungicidal” for a target fungus. That is, the agent kills the target fungal cells and, ideally, is not substantially toxic to mammalian cells. The most common mode of action for antifungal agents is the disruption of the cell membrane by target ergosterol synthesis. The imidazole (e.g., miconazole, ketoconazole and clotrimazole), triazole (e.g., fluconazole), tetrazole (e.g., oteseconazole), and allylamine (e.g., amorolfine, fenpropimorph, butenafine, naftifine, or terbinafine) classes of antifungal agents disrupt ergosterol biosynthesis. By comparison, the polyene class of antifungal agents (e.g., amphotericin B, candicidin, filipin, hamycin, natamycin, nystatin, or rimocidin) bind to ergosterol in fungal cytoplasmic membranes, thus creating pores. Beyond targeting ergosterol, there are a few antifungal agents that target other fungal structures. For example, the echinocandins (e.g., anidulafungin, caspofungin, micafungin and rezafungin) and triterpenoids (e.g., ibrexafungerp) block the synthesis of β (1->3) glucan found in fungal cell walls and the polyoxins and nikkomycins (e.g., nikkomycin Z) target chitin synthesis. Further, there are antifungal agents that block mitosis and act as antimetabolites against fungal processes. For example, griseofulvin is thought to specifically disrupt fungal cell division by interfering with microtubules and atovaquone, a representative of the naphthoquinone drug class, is a semisynthetic antimetabolite for fungal mitochondrial cytochrome.

For the purposes of this invention, pathogenic fungi are intended to include obligate and opportunistic pathogenic fungi that are capable of causing superficial, cutaneous, subcutaneous, systemic, or allergic diseases. Pathogenic fungi may infect healthy and immunocompetent humans and animals, as well as those at risk of fungal infections, in particular, those with reduced immunity or suffering from serious illnesses, i.e., cancer, organ, and hematopoietic stem cell failure, autoimmune diseases, and trauma. In general, pathogenic fungi are heterotrophic, have a chitinous cell wall, plasma membranes containing the sterol ergosterol, and may exhibit varying susceptibility to antifungal agents. In accordance with this invention, mammalian pathogenic fungi are classified as yeasts, molds, or dimorphic fungi. In some aspects of this invention, the synergistic combination of antifungal agent and NK1-anatgonist inhibits the growth of one or more mammalian pathogenic fungi. In other aspects, the synergistic combination of antifungal agent and NK1-anatgonist inhibits the growth of one or more yeasts. In further aspects, the synergistic combination of antifungal agent and NK1-anatgonist inhibits the growth of one or more molds or filamentous fungi. In yet other aspects, the synergistic combination of antifungal agent and NK1-anatgonist inhibits the growth of one or more dimorphic fungi.

Yeasts are solitary cells that reproduce by budding. Yeasts are the causal agents of diseases such as candidiasis and cryptococcosis in humans and animals. The most common etiological agents of candidiasis include Candida spp. such as C. albicans, C. glabrata, C. auris, C. parapsilosis, C. tropicalis, C. krusei, C. dubliniensis, C. guilliermondii, C. kefyr, C. lusitaniae, C. famata and C. rugosa. The most common etiological agents of cryptococcosis are Cryptococcus spp. such as C. neoformans, C. gattii, C. laurentii, C. albidus, C. curvatus, C. uniguttulatus and C. adeliensis. While less prevalent, yeast infections may also be caused by Geotrichum spp. (e.g., G. clavatum), Trichosporon spp. (e.g., T. asahii, T. mucoides, T. mycotoxinivorans), Malassezia spp. (e.g., M. furfur), Saprochaete spp., Kodamaea spp., Rhodotorula spp. (e.g., R. mucilaginosa), Saccharomyces spp. (e.g., S. cerevisiae), Pseudozyma spp., Sporobolomyces spp., Exophiala spp., Lacazia spp., Emmonsia spp., or Wickerhamomyces (Pichia) spp.

In contrast to yeasts, molds or filamentous fungi occur in long filaments known as hyphae, which grow by apical extension. Hyphae can be sparsely septate to regularly septate and possess a variable number of nuclei. Molds are the causal agents of diseases such as aspergillosis and mucormycosis in humans and animals. The most common etiological agents of aspergillosis include Aspergillus spp. such as A. fumigatus, A. flavus, A. terreus, and A. niger. The most common etiological agents of mucormycosis, also known as zygomycosis, include Rhizopus spp. (e.g., Rhizopus oryzae), Mucor spp., Rhizomucor spp., Absidia spp., Lichtheimia spp., Apophysomyces spp., Cunninghamella spp., and Saksenaea spp. Other common infections may be caused by molds such as Scedosporium spp. (e.g., S. apiospermum, S. boydii, S. dehoogii), Fusarium spp. (e.g., F. solani), Paecilomyces spp. (e.g., P. lilacinus, P. variotii), Purpureocillium spp., and dematiaceous fungi such as Alternaria spp., Bipolaris spp., and Curvularia spp.

As used herein, the term “dimorphic fungi” describes fungi that typically grow as a mold in vitro and as either yeast cells or spherules in vivo. Examples of medically important dimorphic fungi include Blastomyces dermatitidis (hyphae and yeast cells), Coccidioides immitis (hyphae and spherules), Histoplasma capsulatum, Paracoccidioides brasiliensis, Penicillium marneffei, Sporothrix schenckii, Emergomyces spp., Talaromyces spp., orEmmonsia-like fungi.

In some aspects, the antifungal agent is a sterol biosynthesis inhibitor, in particular an inhibitor of ergosterol biosynthesis. In other aspects, the antifungal agent is an antifungal agent selected from the group consisting of a polyene antifungal agent, an azole antifungal agent, and an allylamine antifungal agent. In certain aspects, the azole antifungal agent is an imidazole, a triazole, or a thiazole. In one aspect, the imidazole is bifonazole, butoconazole, clotrimazole, econazole, fenticonazole, ketoconazole, luliconazole, miconazole, omoconazole, oxiconazole, sertaconazole, sulconazole, or tioconazole. In another aspect, the triazole is albaconazole, efmaconazole, epoxiconazole, fluconazole, isavuconazole or its prodrug isavuconazonium sulfate, itraconazole, posaconazole, propiconazole, ravuconazole, terconazole, or voriconazole. In a further aspect, the thiazole is abafungin.

Tachykinin NK1 receptor is a member of family 1 (rhodopsin-like) of G protein-coupled receptors and binds to the G_{60 q} protein. An NK1 antagonist is a compound that binds to the NK1 receptor thereby blocking its activity. NK1 antagonists of use in this invention include, but are not limited to:
5-[[(2R,3S)-2-[(1R)-1-[3,5-bis(trifluoromethyl) phenyl]ethoxy]-3-(4-fluorophenyl)-4-morpholinyl]methyl]- 1,2-dihydro-3H-1,2,4-triazol-3-one (aprepitant) as described in US 5,719,147, or in a liquid oral formulation as described in US 2017/0035774, or in an injectable emulsion in a single-dose vial for intravenous use containing 130 mg aprepitant in 18 ml of emulsion as described in U.S. Pat. No. 9,808,465;
[3-{[(2R,3S)-2-[(1R)-1-[3,5-bis(trifluoromethyl) phenyl]ethoxy]-3-(4-fluorophenyl)morpholin-4-yl]methyl}-5- oxo-2H-1,2,4-triazol-1-yl]phosphonic acid (fosaprepitant, MK-0517, or L-758,298) as disclosed, for example, as meglumine salt in U.S. Pat. No. 5,691,336 and as di (cyclohexylamine) salt in US 2016/0355533;
(2S,4S)-4-(4-acetyl-1-piperazinyl)-N- [(1R)-1-[3,5-bis (trifluoromethyl) phenyl] ethyl]-2 (4-fluoro-2-methylphenyl)- N-methyl-1-piperidinecarboxamide (casopitant) as described in U.S. Pat. No. 7,294,630;
(2S)-1-[(3aS,4S,7aS)-4-hydroxy-4-(2-methoxyphenyl)-7,7- diphenyl-1,3,3a,5,6,7a-hexahydroisoindol-2-yl]-2-(2-methoxy phenyl)propan-1one (dapitant);
(2S,3S)-N-(5-tert-butyl-2-methoxybenzyl)-2-(diphenyl methyl)-1-azabicyclo[2.2.2] octan-3-amine (maropitant) as disclosed in U.S. Pat. No. 5,807,867, WO 2005/082416 and EP 3173071;
(2S,3S)-2-benzhydryl-N-[(2-methoxy-5-propan-2-ylphenyl) methyl]-1-azabicyclo[2.2.2]octan-3-amine as described by Evangelista (2001) Curr. Opin. Invest. Drugs 2(10):1441-3;
(2S)-N-{2-[3, 5-bis(trifluoromethyl)phenyl]ethyl}-2-[4- (cyclopropylmethyl)piperazin-1-yl]-N-methyl-2-phenyl acetamide (figopitant);
N-[(2R)-1-[acetyl-[(2-methoxyphenyl)methyl]amino]-3- (1H-indol-3-yl)propan-2-yl]-2 (4-piperidin-1-ylpiperidin-1- yl)acetamide (lanepitant);
2-[3,5-bis(trifluoromethyl)phenyl]-N, 2-dimethyl-N-[4- (2-methylphenyl)-6-(4-methylpiperazin-1-yl) pyridin-3- yl]propanamide (netupitant) as described in U.S. Pat. Nos. 6,297,375, 6,593,472, 6,719,996, or in an oral composition including 300 mg of netupitant and palonosetron hydrochloride in an amount equivalent to 0.5 mg of palonosetron base, referred to as “netupitant-300/palonosetron-0.5” as described in U.S. Pat. No. 8,951,969;
(2R,4S)-4-[(8aS)-6-oxo-1,3,4,7,8,8a-hexahydropyrrolo [1,2-a]pyrazin-2-yl]-N-[(1R)-1-[3,5-bis(trifluoromethyl) phenyl]ethyl]-2-(4-fluoro-2-methylphenyl)-N-methyl piperidine-1-carboxamide (orvepitant) as disclosed in US 2005/0176715 or as crystalline maleate in US 2011/0166150;
(5S,8S)-8-[[(1R)-1-[3,5-bis(trifluoromethyl) phenyl]ethoxy]methyl]-8-phenyl-1,9-diazaspiro[4. 5]decan-2-one (rolapitant) as described in U.S. Pat. No. 7,049,320, or as an injectable form thereof as described in U. S. Pat. No. 9, 101, 615;
3-[(3aR,4R,5S,7aS)-5-[(1R)-1-[3,5-bis(trifluoromethyl) phenyl]ethoxy]-4-(4-fluorophenyl)-1,3,3a,4,5,6,7,7a- octahydroisoindol-2-yl]cyclopent-2-en-1-one (serlopitant) as described in U.S. Pat. No. 7,544,815 and U.S. Pat. No. 7,217,731;
(2S)-N-[(1R)-1-[3,5-bis(trifluoromethyl)phenyl]ethyl]- 2-(4-fluoro-2-methylphenyl)-N-methylpiperazine-1- carboxamide (vestipitant) as described in WO 2001/25219 or as in intravenous formulation having a reduced tendency to cause hemolysis as described in WO 2012/175434;
(2S,3S)-N-[[2-methoxy-5-[5-(trifluoromethyl)tetrazol-1- yl]phenyl]methyl]-2-phenylpiperidin-3-amine (vofopitant), as disclosed by Gardner et al. (1996) Regul. Pept. 65(1):45-53; and
(2S,3S)-N-[[2-methoxy-5-(trifluoromethoxy)phenyl]methyl]-2-phenylpiperidin-3-amine (CP-122721) as described by Obach et al. (2007) Drug Metab. Pharmacokinet. 22(5):336-49.

Illustrative examples of pharmaceutically acceptable salts of basic NK1 antagonists include acid addition salts with mineral acids, such as hydrochloric acid, hydrobromic acid, hydriodic acid, sulfuric acid, phosphoric acid, nitric acid, carbonic acid, phosphoric acid and the like and acid addition salts with organic acids such as formic acid, acetic acid, propionic acid, oxalic acid, malonic acid, succinic acid, fumaric acid, maleic acid, lactic acid, malic acid, tartaric acid, citric acid, ascorbic acid, methanesulfonic acid, ethanesulfonic acid, gluconic acid, aspartic acid, glutamic acid, and the like. Illustrative examples of pharmaceutically acceptable salts of acidic NK1 antagonists such as fosaprepitant include salts with inorganic bases such as alkaline metal or alkaline-earth metal salts, and salts with organic bases such as dicyclohexylamine salts, N-methyl-D-glucamine (meglumine) salts, and salts with amino acids, as described in U.S. Pat. No. 5,691,336.

In certain aspects of this invention, the synergistic combination inhibits the growth of a species of mold such as Aspergillus, Mucor, or Rhizopus. In other aspects, the synergistic combination of posaconazole and rolapitant is used to inhibit the growth of a mammalian fungal pathogen, in particular a human fungal pathogen. In particular aspects, the mammalian fungal pathogen is a species of Candida, Aspergillus, Cryptococcus, Mucor, or Rhizopus. In other aspects, the Candida is a species selected from the group of C. glabrata, C. auris, C. albicans, C. dubliniensis, and C. parapsilosis.

The combination of antifungal agents with NK1 antagonists offers a novel therapeutic strategy to increase and, in some cases, restore the utility of antifungal agents such as antifungal triazoles against difficult to treat pathogens. Accordingly, in addition to in vitro applications described herein, the present invention also provides for in vivo use of the synergistic combination to inhibit the growth of pathogenic fungi and treat a fungal infection, i.e., an abnormal and/or undesired presence of a fungus in or on a subject. In the context of treatment, a synergistic effective amount of an NK1 antagonist and antifungal agent is administered to a subject in need thereof to treat the subject's fungal infection. A “subject in need” of treatment for a particular condition can be a subject having that condition, diagnosed as having that condition, or at risk of developing that condition. Preferably, the subject is a mammal. The mammal can be a human, non-human primate, mouse, rat, dog, cat, horse, or cow, but is not limited to these examples. Mammals other than humans can be advantageously used as subjects that represent animal models of conditions described herein. A subject can be male or female.

In some aspects, the subject is immunocompromised. In other aspects, the subject is infected with HIV/AIDS or has cancer, e.g., acute myeloid leukemia or acute lymphoid leukemia. In some aspects, the subject has neutropenia or lymphopenia. In aspects, the subject is undergoing or has undergone cancer chemotherapy treatment, corticosteroid treatment, or TNF inhibitor treatment. In other aspects, the subject is an organ transplant recipient or a hematopoietic stem-cell transplant recipient. In further aspects, the subject has graft-versus-host disease.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g., a condition or disease described herein. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

Evaluating subjects for fungal infections and assessing efficacy of treatment includes multiple modalities of diagnostic testing, including: radiological assessments including CT scanning of the chest, sinuses, and abdomen; detecting the level of fungal load in a biological sample (for example, a tissue biopsy, blood test, or urine test); detecting the level of a surrogate marker of the fungal infection in a biological sample; detecting symptoms associated with the infection; or detecting immune cells involved in the immune response typical of fungal infections (for example, detection of antigen specific T cells or antibody production).

In some aspects, the fungal infection is superficial, locally invasive, or disseminated throughout the subject. In certain aspects, the fungal infection is a cutaneous infection, lung infection, sinus infection, central nervous system infection, brain infection, eye infection, heart infection, kidney infection, gastrointestinal tract infection, stomach infection, pelvic infection, blood infection, or a combination thereof. In particular aspects, the fungal infection is a fungal disease or condition selected from the group of allergic bronchopulmonary aspergillosis, allergic sinusitis, azole-resistant A. fumigatus, aspergilloma, pulmonary aspergillosis, invasive aspergillosis, cutaneous aspergillosis, fusariosis, rhinocerebral mucormycosis, pulmonary scedosporiosis, mucormycosis, disseminated mucormycosis, abdominal-pelvic mucormycosis, gastric mucormycosis, cutaneous mucormycosis, infectious keratitis and/or endopthalmitis, thrush (caused by C. albicans); cryptococcosis (caused by Cryptococcus); and histoplasmosis, blastomycosis, paracoccidioidomycosis, oral geotrichosis, Rhodotorula infection or a combination thereof.

The synergistic composition described herein can be administered to a subject having or diagnosed as having a fungal infection. A variety of means for administering the synergistic composition to a subject are known to those of skill in the art. Such methods can include, but are not limited to oral, parenteral, intravenous, intramuscular, subcutaneous, transdermal, intradermal, airway (aerosol), pulmonary, cutaneous, ocular, corneally, or by injection. Administration can be local or systemic. The route of administration may be via systemic administration, oral administration, intravenous administration, topical administration, transdermal administration or parenteral administration.

The synergistic composition as described herein, i.e., a composition comprising at least one NK1 antagonist and at least one antifungal agent, can further include a pharmaceutically acceptable carrier. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like.

The term “carrier” in the context of this invention refers to a diluent, adjuvant, excipient, or vehicle in admixture which the active agents. Such carriers can be sterile liquids, such as water and oils, including those of petroleum, animal, vegetable or synthetic origin, such as peanut oil, soybean oil, mineral oil, sesame oil and the like. Water is a preferred carrier when the composition is to be administered intravenously. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents.

The synergistic composition can take the form of solutions, suspensions, emulsions, microemulsions, tablets, pills, capsules, powders, liquid syrups, soft gels, sustained-release formulations, eye drops, creams, foams, gels, hydrogels, lotions, ointments, liposome-containing formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. The formulation should suit the mode of administration and preferably presented in unit dosage form prepared according to conventional techniques well known in the pharmaceutical industry.

A variety of known controlled-or extended-release dosage forms, formulations, and devices can also be used in the methods of this invention. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1. These dosage forms can be used to provide slow or controlled-release of one or more active ingredients using, for example, hydroxypropylmethyl cellulose, other polymer matrices, gels, permeable membranes, osmotic systems (such as OROS® sold by Alza Corporation, Mountain View, CA), or a combination thereof to provide the desired release profile in varying proportions.

The synergistic composition may additionally contain other auxiliary components conventionally found in pharmaceutical compositions. The pharmaceutical compositions may contain additional compatible pharmaceutically active antipruritics, astringents, local substances such as anesthetics or anti-inflammatory agents, or may contain additional materials such as buffers, dyes, preservatives, antioxidants, sunscreens, thickeners and stabilizers or combinations thereof used to physically formulate the various dosage forms of the compositions of the present invention.

However, such materials, when added, should not unduly interfere with the biological activity of the active ingredients.

The dosage depends on the severity and responsiveness of the disease state to be treated, and the course of treatment may last from several days to several months, or until a cure is reached or a diminution of the disease state is achieved. The optimal dosing regimen may be calculated from measurements of drug accumulation in the patient's body. The administering physician can readily determine the optimal dosage, method of administration and repetition rate. The optimal dosage may vary according to the relative potency of the composition and may generally be estimated based on toxic and therapeutic effects expressed as the ratio LD₅₀/ED₅₀. Compositions and methods that exhibit large therapeutic indices are preferred. A therapeutically effective dose can be estimated initially from cell culture assays. Also, a dose can be formulated in animal models to achieve a circulating plasma concentration range that includes the IC₅₀ (i.e., the concentration of the active compound, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

In some aspects, an effective dose of a composition described herein, e.g., a composition comprising at least one NK1 antagonist and at least one antifungal agent, can be administered to a subject once. In other aspects, an effective dose a composition described herein, e.g., a composition comprising at least one NK1 antagonist and at least one antifungal agent, can be administered to a subject repeatedly. In certain aspects, an effective dose a composition described herein, e.g., a composition comprising at least one NK1 antagonist and at least one antifungal agent, can be administered to a subject daily. For systemic administration, subjects can be administered an effective amount of a composition of this invention, such as, e.g., 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

The dosage of a composition can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the active compound. The desired dose or amount of activation can be administered at one time or divided into subdoses, e.g., 2-4 subdoses and administered over a period of time, e.g., at appropriate intervals through the day or other appropriate schedule. In some aspects, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more.

The synergistic composition may be administered simultaneously, i.e., the NK1 antagonist and antifungal agent are administered together, e.g., in a single dosage form, or the components of the synergistic composition may be administered consecutively, e.g., as two dosage forms (which may be the same or different), one after the other.

For use in the therapeutic, kits and articles of manufacture are also provided. In some aspects, the kit includes a container including a synergistic composition comprising an NK1 antagonist and an antifungal agent. In other aspects, the kit includes a container including an NK1 antagonist and a separate container including an antifungal agent. In some aspects, such kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. In some aspects, the containers are formed from a variety of materials such as glass or plastic.

The articles of manufacture may contain packaging materials. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment.

A kit will typically include one or more additional containers, each with one or more of various materials (such as reagents, optionally in concentrated form, and/or devices) desirable from a commercial and user standpoint for use of a synergistic composition described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included.

In a further aspect of this invention, the synergistic composition described herein is used as a disinfectant to remove difficult to kill fungal organisms from surfaces in laboratory or medical settings as well as apparel (e.g., clothing or footwear), flooring and rugs/mats, and articles worn by animals (e.g., leashes, bridles, cinches, saddles, blankets, booties, fomites, and horseshoes). The surface disinfectant of this invention includes a synergistic amount of an NK1 antagonist and an antifungal agent in admixture with a carrier and/or excipient. The use of particular carriers or excipients can also function in the invention to increase the penetration of the substrate, the rate of penetration, the thoroughness of coverage, etc. Carriers or excipients can also be used to cause fungal spores to end dormancy and begin germination, thus making the spore more susceptible to treatment with the synergistic composition. Carriers or excipients of use in the disinfectant of this invention include, but are not limited to, water, a detergent, an oil, a glycol, an alcohol, another polar solvent, combinations thereof, or any other liquid or solid that does not have a negative effect on the active materials or surfaces to be treated. In some aspects, the disinfectant includes a preservative.

The disinfectant may be delivered in the form of an aerosol, spray, fog, powder, wipe, insertion, or impregnation of a surface or substrate with the synergistic composition. For example, the composition may be present within a solid matrix or substrate such as a non-woven (e.g., cotton) wipe or viscose or polypropylene-based interleaved sheet. In this manner the composition of the invention is impregnated in the solid substrate. Alternatively, the matrix may be provided in combination with, e.g., dispersed within, a liquid or gel matrix, such as an emulsion or emollient composition. Additionally, the composition may be provided as a liquid spray. In this form, it will be understood that additional carriers may not necessarily be required as the formulation is already in liquid form (with the purified/sterilized water acting as the carrier). However, optional excipients may be added, for example to add fragrance, alter the viscosity of the liquid etc. The disinfectant also be provided in the form of liquid gel tablets or capsules, for later delivery or introduction by squeezing/rupturing. Additional excipients may be required to provide a gel-like matrix or the formulation may simply be encased in a gel tablet casing.

In addition to medically relevant fungal pathogens, the synergistic composition of this invention may also be used to inhibit the growth of agriculturally relevant pathogens. In this respect, the present invention also provides for methods and compositions to inhibit the growth of plant fungal pathogens such as Alternaria spp., Fusarium spp., Cochliobolus spp., Albugo spp., Pythium spp. Rhizoctonia spp., Sclerotinia spp., Botrytis spp., Colletotrichum spp., Phytophthora spp., Puccinia spp., Uromyces spp., Septoria spp., and Verticillium spp. The instant composition may be provided in the form of conventional fungicidal composition such as a solution, aerosol, spray, fog, or powder and applied to the roots, stems or leaves or a plant in need of treatment.

What is claimed is:

1. A method for inhibiting the growth of a pathogenic mold comprising contacting the pathogenic mold with a synergistically effective amount of an NK1 antagonist and an antifungal agent thereby inhibiting the growth of the pathogenic mold.

2. The method of claim 1, wherein the antifungal agent is a sterol biosynthesis inhibitor.

3. The method of claim 2, wherein the sterol biosynthesis inhibitor is a triazole.

4. A method for inhibiting the growth of a mammalian fungal pathogen comprising contacting the mammalian fungal pathogen with a synergistically effective amount of posaconazole and rolapitant.

5. The method of claim 4, wherein the mammalian fungal pathogen is a species of Candida, Aspergillus, Cryptococcus, Mucor, or Rhizopus.

6. The method of claim 5, wherein the Candida species comprises Candida glabrata, Candida auris, Candida albicans, Candida dubliniensis, and Candida parapsilosis.

7. A surface disinfectant comprising a synergistic amount of an NK1 antagonist and an antifungal agent in admixture with a carrier or excipient.
